(19) United States
(12) Patent Application Publication
(10) Pub. No.: US 2007/0032848 A1
(43) Pub. Date: Feb. 8, 2007

(54) Elastic therapeutic wrap

(76) Inventor: Cliff Bridges, Greensboro, NC (US)

Correspondence Address: Wood, Phillips, Katz, Clark & Mortimer, Citigroup Center, Suite 3800, 500 West Madison Street, Chicago, IL, 60661-2511, US

(21) Appl. No.: 11/197,058

(22) Filed: Aug. 4, 2005

(51) Int. Cl. A61F 7/00

(52) U.S. Cl. 607/114

(57) ABSTRACT

The present invention is directed to an elastic therapeutic wrap to administer therapeutic relief to a body part to be treated, and more specifically is directed to a low cost elastic wrap, including a nonwoven fabric, and at least one receptacle for receiving a therapeutic body pack, such as a heat pack, cold pack, or herbal pack that is readily replaceable and interchangeable. The therapeutic wrap includes a first nonwoven fabric exhibiting stretch and recovery in the cross-direction, a second nonwoven fabric exhibiting stretch and recovery in the cross-direction, at least one open-ended receptacle formed between the first and second nonwoven fabrics for receiving a therapeutic body pack, and a fastener for fastening the wrap to a body part to be treated.

### TECHNICAL FIELD

The present invention is generally related to a therapeutic body wrap, and more particularly relates to a body wrap formed of a nonwoven fabric and having a receptacle for receiving a therapeutic body pack.

### BACKGROUND OF THE INVENTION

Therapeutic wraps are known in the art for use in relieving aches and pains in the muscles and joints. In treating muscle injuries, heat therapy or cold therapy is utilized on the injury site to promote blood flow to the area and/or reduce swelling. Various sizes of therapeutic wraps are available to conform to different body parts, such as the neck, back, knee, or ankle. The prior art includes therapeutic wraps that utilize heat packs or cold packs for delivering hot or cold therapy directly to the site of injury.

Disposable therapeutic wraps are known in the art, wherein a plurality of heated disks are affixed and enclosed within the wrap, which is eventually disposed of after a single use. Further, therapeutic wraps utilizing multiple layers of material are known, wherein woven insulating layers and aluminized thermal reflective layers are incorporated, adding to the overall manufacturing cost of the wrap. Further still, other therapeutic wraps utilize multiple straps and fasteners for proper positioning of the wrap, as well as proper positioning of a heat pack or cold pack, which may be removable and repositionable at one or more locations along the length of the wrap. Such therapeutic wraps as disclosed in the prior art are typically costly, non-reusable, and/or difficult to use due to an excessive number of straps and fasteners.

In view of the above disadvantages, there remains a need for an improved therapeutic wrap. It is therefore an object of the present invention to provide a low cost, stretchable, nonwoven therapeutic wrap. It is further an object of the invention to provide a low cost, reusable therapeutic wrap, and yet another object of the present invention to provide a therapeutic wrap that is easily positioned and fastened about the body part to be treated.

### SUMMARY OF THE INVENTION

The present invention is directed to an elastic therapeutic wrap to administer therapeutic relief to a body part to be treated, and more specifically is directed to a low cost elastic wrap, including a nonwoven fabric, and at least one receptacle for receiving a therapeutic body pack, such as a heat pack, cold pack, or herbal pack that is readily replaceable and interchangeable.

In accordance with the present invention, the therapeutic wrap includes a first nonwoven fabric exhibiting stretch and recovery in the cross-direction, a second nonwoven fabric exhibiting stretch and recovery in the cross-direction, at least one open-ended receptacle formed between the first and second nonwoven fabrics for receiving a therapeutic body pack, and a fastener for fastening the wrap to a body part to be treated. The first nonwoven fabric typically has a pre-determined length that is equal to less than the length of the second nonwoven fabric. The elastic therapeutic wrap may further include a first end portion, a second end portion, and a middle portion, wherein the first end portion and the second portion respectively include fastening members that can be engaged once the therapeutic wrap is positioned about a user's body part to be treated. In addition, the middle portion may have a width that is greater than the widths of the first and second end portions.

In one embodiment, the middle portion of the therapeutic wrap will typically include one or more open ended receptacles, formed between the first and second nonwoven fabrics, that can receive one or more heat packs, cold packs, herbal packs, and the like. In one embodiment, the therapeutic wrap includes a single receptacle that may extend the entire length of the wrap, or extend the length of the middle portion of the wrap. In another embodiment, the therapeutic wrap includes two or more receptacles that may be positioned in a side-by-side relationship along the entire length of the wrap, or positioned in two or more separate locations anywhere along the length of the wrap.

The first and second end portions define the length of the therapeutic wrap, with the end portions respectively including fastening members for affixing the wrap about the body. In an illustrative embodiment, the wrap of the present invention utilizes a hook and loop fastening member; however, the present invention also contemplates use of other fasteners, including ties, eye hooks, magnetic closures, and the like.

Other features and advantages of the present invention will become readily apparent from the following detailed description, the accompanying drawings, and the appended claims.

### BRIEF DESCRIPTION OF THE DRAWINGS

FIG. 1a is a diagrammatic view illustrating a first embodiment of the therapeutic body wrap of the present invention;

FIG. 1b is a diagrammatic view illustrating an alternative embodiment of the therapeutic body wrap of the present invention; and

FIG. 2 is a diagrammatic view of a suitable apparatus for imparting a pattern or image to a fabric for forming a therapeutic wrap in accordance with the present invention.

### DETAILED DESCRIPTION

While the present invention is susceptible of embodiment in various forms, there is shown in the drawings and will hereinafter be described presently preferred embodiments, with the understanding that the present disclosure is to be considered as an exemplification of the invention, and is not intended to limit the invention to the specific embodiments illustrated.

The present invention is directed to an elastic, therapeutic wrap to administer therapeutic relief to a body part, such as the lumbar or sacral regions of the lower back, and more specifically is directed to a low cost elastic wrap including a nonwoven fabric, and at least one receptacle for removably receiving therapeutic body packs. Accordingly, the therapeutic wrap includes a first elastic nonwoven fabric exhibiting stretch and recovery in the cross-direction, a second elastic nonwoven fabric exhibiting stretch and recovery in the cross-direction, at least one open-ended receptacle formed between the first and second nonwoven fabrics for receiving a therapeutic body pack, and a fastening arrangement for fastening the wrap about a user's body part.

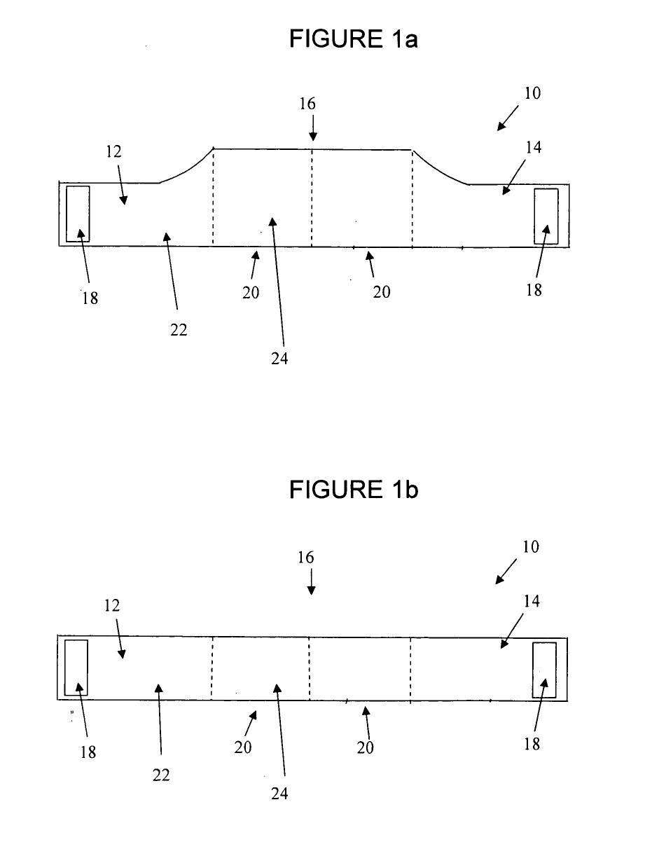

FIGS. 1a and 1b are illustrative embodiments of the therapeutic body wrap of the present invention. The elastic nonwoven fabric for formation of the therapeutic wrap 10 may be die-cut to a given length, wherein the wrap 10 has a first end potion 12, a second end portion 14, and middle portion 16 that together define the length of the wrap 10. Further, the middle portion 16 of the wrap 10 may have a width consistent with and generally equal to, the width of the first and second end portions 12 and 14, or the width of the middle portion 16 may be greater than the width of the first and second end portions 12 and 14. FIG. 1a is an illustrative embodiment wherein the middle portion 16 of the wrap has a width greater than the width of the first and second end portions 12 and 14. FIG. 1b is an illustrative embodiment wherein the wrap 10 has a uniform width throughout, i.e., the middle portion 16 of the wrap is generally equal to the width of each end portion 12 and 14.

As further shown in FIGS. 1a and 1b, the middle portion 16 of the therapeutic wrap 10 includes one or more open ended receptacles 20, defined by and formed between first and second nonwoven fabrics 22 and 24, that can respectively receive one or more heat packs, cold packs, herbal packs, and the like. The therapeutic wrap 10 may include a single receptacle that extends substantially the entire length of the wrap 10, or extend substantially the length of the middle portion 16 of the wrap 10. Alternatively, the therapeutic wrap 10 may include two or more receptacles 20 that may be positioned in a side-by-side relationship and extend along substantially the entire length of the wrap 10, or positioned in two or more separate, spaced apart locations anywhere along the length of the wrap 10. The open-ended receptacles 20 formed by the first and second nonwoven fabrics 22 and 24 can be fashioned by stitch bonding, adhesive bonding, thermal bonding, or by a combination thereof. The formed open-ended receptacles 20 are designed to removably receive a variety of replaceable therapeutic packs. In the illustrated embodiments, the length of second nonwoven fabric 24 corresponds to the combined length of the receptacles 20.

Heat packs suitable for use with the body wrap of the invention include reusable heat packs as taught in U.S. Pat. No. 4,077,390, issued on Mar. 7, 1978 in the name of inventor Stanley, et al., U.S. Pat. No. 4,880,953, issued on Nov. 14, 1989 in the name of inventor Manker, and U.S. Pat. No. 6,537,309, issued on Mar. 25, 2003 in the name of inventor Sharma, et al., as well as moist heat packs as disclosed in U.S. Pat. No. 5,447,531, issued on Sep. 5, 1995 in the name of inventor Wood, and expandable heat packs described in U.S. Pat. No. 6,640,801, issued on Nov. 4, 2003 in the name of inventor Sabin, et al., all of which are hereby incorporated by reference. Alternatively, the one or more receptacles within the wrap may receive at least one cold pack. Cold therapy may be administered utilizing a reusable cold pack as disclosed in U.S. Pat. No. 4,865,012, issued on Sep. 12, 1989 in the name of inventor Kelley, and U.S. Pat. No. 6,099,588, issued on Aug. 8, 2000 in the name of inventor McDevitt, et al., hereby incorporated by reference. In addition, disposable instant cold packs can be utilized, wherein the packs are kneaded to activate a cooling chemistry as taught, for example in U.S. Pat. No. 6,438,965, issued on Aug. 27, 2002 in the name of inventor Liao, also hereby incorporated by reference. Further still, one or more herbal packs including oils and botanicals may be utilized in combination with or apart from one or more heat or cold packs. Herbs such as peppermint, spearmint, cinnamon, chamomile, lavender, saw palmetto berry, lemon grass, valerian or yellow duck root, yarrow, rosemary, and white willow are known as antispasmodics, pain relievers, relaxants, and circulation boosters, and may be inserted into one or more receptacles of the wrap, or combined with a heat therapy pack within the same receptacle to deliver an aromatic and/or herbal form of relief in combination with heat therapy.

In addition, the first end portion 12 and second portion 14 of the therapeutic wrap 10 may respectively include one or more releasable fastening members fastener 18 that can be cooperatively engaged once the wrap 10 is positioned about a user's body to removably secure the wrap into place. In one embodiment, the first end portion 12 has a loop fastening member permanently affixed thereto, and the second end portion 14 has a complementary hook member permanently affixed, whereby the hook and loop fastener can be initially engaged, disengaged, and re-engaged to adjust the fit of the therapeutic wrap 10. Hook and loop fasteners are commercially available under the Velcro® tradename. Optionally, the wrap may utilize alternate fasteners, such as a magnetic closure system, eye hooks, and the like.

Additionally, the therapeutic body wrap of the present invention may include one or more additives, which may be incorporated within the polymer, as a melt additive from which the nonwoven fabric of the wrap is formed, or may be topically applied to the fabric. Suitable additives include, but are not limited to dyes, fragrances, antimicrobials, emollients, thermochromics, and the combinations thereof. Further, the therapeutic body wrap of the present invention can be configured for reuse, such that the wrap may be repeatedly laundered to rid the wrap of instilled body odors, exfoliated skin cells, and residues deposited onto the therapeutic wrap fabric by herbal packs.

The present invention can be formed in accordance with the methods disclosed in U.S. Pat. No. 3,485,706, issued on Dec. 23, 1969 in the name of inventor Evans, hereby incorporated by reference, wherein a process for effecting hydroentanglement of nonwoven fabrics is described, and U.S. Pat. No. 5,098,764, issued on Mar. 24, 1992 in the name of inventor Drelich, et al., hereby incorporated by reference, wherein a nonwoven fabric having unique characteristics is disclosed that permits use of the fabric in a wide variety of applications. A method and apparatus for manufacturing the fabric are also disclosed in the Drelich et al. patent, including a hydroentanglement (sometimes referred to as spun-laced) process by which a precursor nonwoven web of fibers is subjected to hydroentanglement on a forming surface to impart a rectilinear pattern to the web. The present invention can be formed in accordance with the teachings of aforementioned U.S. Pat. No. 5,098,764, and as appropriate, reference will be made to this patent in connection with the present disclosure. Further, U.S. Pat. No. 5,670,234, issued on Sep. 23, 1997 in the name of inventor Suehr, et al., U.S. Pat. No. 5,674,587, issued on Oct. 7, 1997 in the name of inventor James et al., and U.S. Pat. No. 5,827,597, issued on Oct. 27, 1998 also in the name of inventor James, et all., all hereby incorporated by reference, disclose a topographical support member which may be employed for imparting a pattern to a nonwoven fabric during hydroentanglement, with the resultant fabric again having desirable properties which lend it for use in many different applications.

The above-referenced patents may be used for manufacture of the nonwoven therapeutic wrap of the present invention, wherein the nonwoven fabric exhibits elastic characteristics, that is, extensibility and recovery, in the cross-direction of the fabric. Nonwoven fabrics typically exhibit a machine-direction and a cross-direction, that is, with reference to the direction which extends along the length of the fabric, i.e., the direction in which it is manufactured (the machine-direction), and the direction of the fabric which extends perpendicularly to the machine-direction, typically across the width of the fabric (the cross-direction). The data presented in Table 1 is exemplarily of the stretch and recovery performance of a nonwoven fabric suitable for use in the therapeutic wrap of the present invention, wherein the nonwoven fabric has a cross-direction extensibility of at least 50% and a cross-direction recovery of at least 90%.

The orientation of fibers or filaments of a nonwoven fabric, with respect to the cross-direction and machine-direction, can significantly impact upon the resultant properties and characteristics of the fabric. As will be recognized by those familiar with the art, a nonwoven fabric formed from a precursor web formed by “100% in-line card” refers to a web formed entirely from carded fibers, wherein all of the fibers are principally oriented in the machine direction of the web. In contrast, a precursor web formed by “all crosslap” refers to a fibrous web wherein the fibers or filaments have been formed by crosslapping a carded web so that the fibers or filaments are oriented at an angle relative to the machine direction of the resultant web. A precursor web can be formed by “one-half crosslap, one-half card”, wherein one-half of the basis weight of the precursor web includes a carded fiber web, and one-half of the basis weight includes a crosslap fiber web. A precursor web may further be formed with combinations of in-line carded fibers with machine direction orientation, and cross-directional randomized fibers.

Manufacture of a nonwoven fabric for use in accordance with the present invention is initiated by providing a precursor nonwoven web which may include staple length textile fibers, including synthetic fibers, natural fibers, and blends thereof, as well as continuous filaments. Suitable fibers or filaments include, but are not limited to mono-components, multi-components, co-polymeric fibers or filaments, elastomeric fibers or filaments, and blends thereof.

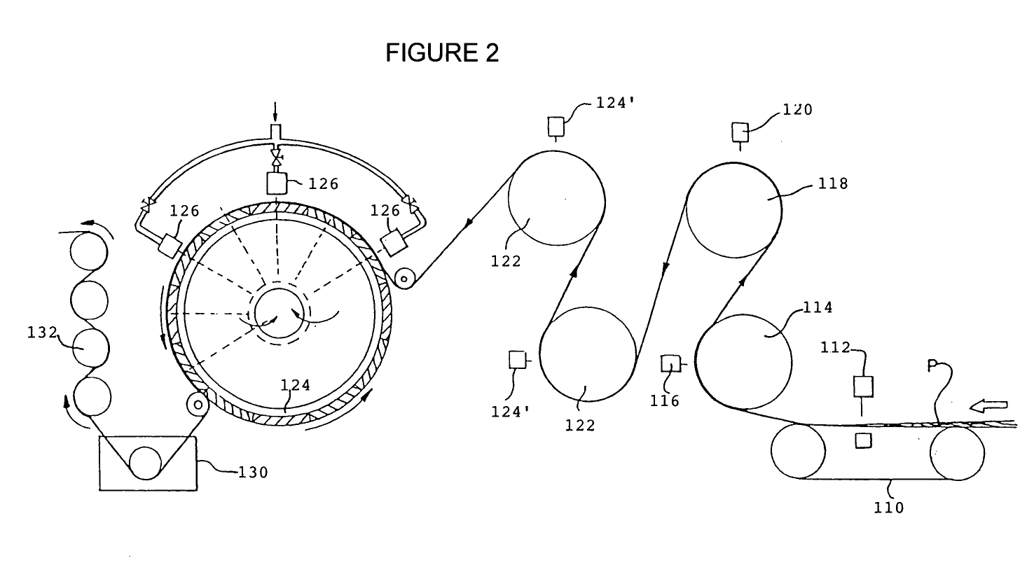

A precursor web is formed in accordance with conventional carding and crosslapping techniques prior to subjecting the web to hydroentanglement, and binder application, in accordance with the present invention. In the preferred form, the precursor web is subjected to hydroentanglement prior to patterned hydroentanglement of the web in accordance with the above-referenced patents. FIG. 2 diagrammatically illustrates the apparatus for practicing a suitable method for imparting a pattern or image into the nonwoven fabric. As shown, a precursor web P is initially received on a belt 110, on which the web P is subjected to the first of a series of hydroentangling treatments. Hydroentanglement of the web P being carried by belt 110 is affected by nozzle assembly 112, which is operated to discharge high-pressure columnar jets or streams of liquid, typically water.

The precursor web is then moved to an entangling drum 114 having a 100 mesh screen surface. Nozzle assembly 116 is configured like nozzle assembly 112, and effects further entanglement of the precursor web. The precursor web is then moved to a processing drum 118, also having a 100 mesh screen, at which it is subjected to entangling by nozzle assembly 120, configured like nozzle assemblies 112 and 116. The precursor web is then received about two successive processing drums 122 having micro-porous shells, each having a respective nozzle assembly 124′, 124′, configured as the above-described nozzle assemblies. The precursor web has been subjected to entanglement energy generally on the order of 0.05 to 0.30 horsepower-hour per pound, with the web now directed to a hydroentangling apparatus for patterned hydroentanglement of the precursor web.

The illustrated embodiment of the final entangling apparatus is in accordance with above-referenced U.S. Pat. No. 5,098,764, and includes a processing drum 124 (sometimes referred to as an ITD, or image transfer device) which receives the precursor web P and which typically imparts a final pattern to the web. While the use of an image transfer device is the presently contemplated method for imparting a pattern or image to the nonwoven fabric, it is also contemplated that embossed screens or belts, perforated metal drums, as well as other image and patterning devices may be utilized in place of an image transfer device. The web is subjected to hydroentanglement from three nozzle assemblies, designated 126. Each of the nozzle assemblies is preferably configured in accordance with the above-described nozzle assemblies.

It is within the purview of the present invention that the first and second nonwoven fabrics of the therapeutic body wrap may include similar or dissimilar performance or aesthetic characteristics. For example, the first nonwoven fabric 22 as illustrated in FIG. 1a may exhibit extensibility and recovery performance similar to, dissimilar from, the second nonwoven fabric 24. Further, the first nonwoven fabric 22 may include a pattern or image that is similar to, or dissimilar from the second nonwoven fabric 24. In addition, the first nonwoven fabric 22 may include an additive that is similar to, or dissimilar from, an additive included in the second nonwoven fabric 24.

Subsequent to patterned hydroentanglement, the web receives a substantially uniform application of a polymeric binder composition at an application station 130. The web is then directed over a series of drying cans 132, typically operated at 310 degrees F., with manufacture of the nonwoven fabric thus completed. In addition to the use of binders to impart elasticity, fibers or filaments exhibiting various degrees of elasticity may also be utilized, in whole or in part, to impart elastic characteristics to the fabric, wherein such elastomeric fibers or filaments may include mono-component or multi-component fiber or filaments, copolymeric fibers or filaments, and the combinations thereof.

From the foregoing, it will be observed that numerous modifications and variations can be effected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

TABLE 1

| Averaqe | Sect Wgt | Bulk | MDT | MDE | CDT | CDE | MD Tear | CD Tear | CD Stretch | CD Recovery | MD Softness |
|---|---|---|---|---|---|---|---|---|---|---|---|
| Data Points | (osy) | (in/1 ply) | (lbs) | (%) | (grms) | (%) | (grms) | (grms) | (%) | (%) | (gms) |
| Average | 2.38 | 0.022 | 69.4 | 28.9 | 33.9 | 137.2 | 570 | 880 | 56.3 | 95.7 | 106.2 |

What is claimed is:

1. A therapeutic body wrap, comprising:
a first nonwoven fabric exhibiting extensibility and recovery in a cross-direction;
a second nonwoven fabric affixed in co-extensive relationship with at least a portion of the first nonwoven fabric, said second nonwoven fabric exhibiting extensibility and recovery in a cross-direction;
at least one open ended receptacle defined between the first and second nonwoven fabric for receiving a therapeutic body pack; and
at least one fastener for fastening the wrap to a body part.

2. A therapeutic body wrap as in claim 1, wherein the body wrap comprises a first end portion, a second end portion, and a middle portion together defining a length of the body wrap, the receptacle being defined by said middle portion of the body wrap.

3. A therapeutic body wrap as in claim 2, wherein the middle portion has a width greater than a width of the first and second end portions.

4. A therapeutic body wrap as in claim 1, wherein the first nonwoven fabric is greater in length than the second nonwoven fabric.

5. A therapeutic body wrap as in claim 1, wherein the open-ended receptacle defined between the first and second nonwoven fabrics is formed by mechanically or thermally joining the first and second nonwoven fabrics.

6. A therapeutic body wrap as in claim 5, wherein the receptacle is formed by stitching which joins the first and second nonwoven fabrics.

7. A therapeutic body wrap as in claim 5, wherein the receptacle is formed by thermal bonding which joins the first and second nonwoven fabrics.

8. A therapeutic body wrap as in claim 1, wherein the therapeutic body pack is selected from the group including a heat pack, a cold pack, and an herbal pack.

9. A therapeutic body wrap as in claim 1, wherein the nonwoven fabrics are each comprised of staple fiber, continuous filaments, or a combination thereof.

10. A therapeutic body wrap as in claim 1, wherein the nonwoven fabrics are each apertured.

11. A therapeutic body wrap as in claim 1, wherein the first nonwoven fabric comprises one or more three-dimensional images.

12. A therapeutic body wrap as in claim 1, wherein the second nonwoven fabric comprises one or more three-dimensional images.

13. A therapeutic body wrap as in claim 1, wherein the first nonwoven fabric has a cross-direction extensibility of at least 50% and cross-direction recovery of at least 90%.

14. A therapeutic body wrap as in claim 1, wherein the second nonwoven fabric has a cross-direction extensibility of at least 50% and a cross-direction recovery of at least 90%.

15. A therapeutic body wrap, comprising:
a first nonwoven fabric exhibiting extensibility and recovery in a cross-direction thereof;
a second nonwoven fabric exhibiting extensibility and recovery in a cross-direction thereof, the second nonwoven fabric being in coextensive relationship with at least a portion of the first nonwoven fabric,
the first and second nonwoven fabrics together defining a length of the body wrap, with the cross-directions of the nonwoven fabrics oriented along the length of the body wrap;
the first and second nonwoven fabrics being joined to each other to define at least one receptacle therebetween; and
at least one fastening element for joining end portions of the body wrap for holding the body wrap in place on a body part of a user to be treated.

16. A therapeutic body wrap in accordance with claim 15, wherein:
the body wrap includes a middle portion having a width greater than the end portions of the body wrap.

17. A therapeutic body wrap in accordance with claim 16, wherein:
the receptacle defined by the first and second nonwoven fabrics is provided in the middle portion of the body wrap.

18. A therapeutic body wrap in accordance with claim 15, wherein:
the first nonwoven fabric has a length greater than a length of the second nonwoven fabric, so that the first nonwoven fabric provides the end portions of the body wrap which can be joined by the fastening element.

19. A therapeutic body wrap in accordance with claim 15, wherein:
each of the first and second nonwoven fabrics exhibit cross-direction extensibility of at least 50%, and cross-direction recovery of at least 90%.
